(19) United States
(12) Patent Application Publication
(10) Pub. No.: US 2020/0009302 A1
(43) Pub. Date: Jan. 9, 2020

(54) UNIVERSAL SUCTION AIRFLOW CONTROL DEVICE

(71) Applicant: CASEY PYLE, CAMARILLO, CA (US)

(72) Inventor: CASEY PYLE, CAMARILLO, CA (US)

(21) Appl. No.: 16/298,976

(22) Filed: Mar. 11, 2019

Related U.S. Application Data
(60) Provisional application No. 62/640,811, filed on Mar. 9, 2018.

(51) Int. Cl. A61M 1/00

(52) U.S. Cl. CPC A61M 1/0035; A61M 1/0086

(57) ABSTRACT

A universal suction control device is disclosed that can be arranged in relation to surgical tubing to control the amount of air passing through the tubing. The suction control devices are arranged for easy, point-of-care control of suction while minimizing excessive noise pollution from suction during surgery. One suction control device comprises a mounting mechanism for mounting on surgical suction tubing. A first blocking portion is included for engaging the suctional tubing to block the passage of suction air through the tubing. A control surface is also included that is movable to control the engagement of said blocking portion with said surgical suction tubing to control the amount of air passing through said surgical suction tubing.

This application claims the benefit of U.S. Provisional Patent Application Ser. No. 62/640,811 filed on Mar. 9, 2018.

### BACKGROUND OF THE INVENTION

### Field of the Invention

The present invention is directed to tools used in surgery and in particular, suction tools used in surgery.

### Description of the Related Art

Suction is invariably used in most surgical procedures and is essential for maintaining visualization and removal of unwanted fluid and debris during cases. It is also a source of significant noise pollution during surgery, averaging at 67 dB and up to 74.1 dB, which is in excess of World Health Organization (WHO) recommended work environment level of 35 dB and as low as possible in patient care areas. The noise level of operating rooms as a whole has been shown to exceed OSHA and NIOSH standards for acoustic safety and could be linked with operator distraction or miscommunication during surgery. The white noise of suction can cause an effect known as “masking”, where it becomes difficult to distinguish between multiple similar auditory signals.

Occupational exposures to noise as low as 55 dB has been associated with hypertension, headaches, and fatigue. Constant exposure to noise can lead to a perpetual state of autonomic agitation, catecholamine and stress hormone release and therefore higher incidences of peptic ulcer disease, asthma, and colitis. Studies have shown the instruments used in the operating room to be a significant source of noise during surgery and have given recommendations to find ways to minimize excess/ambient noise.

Suction used during surgery is generally run constantly during the case and produces constant noise. It is inconvenient and inefficient to have a circulating nurse turn suction on and off when it is needed as its use is often unpredictable, sporadic, and brief. A suction tip with an on/off switch is a solution, however, some cases require multiple types of suction tips and surgeon preference for suction tips is variable. To date, only a single series suction tip with an on/off switch incorporated into its design exists. Producing specialized versions of all available suction tips is cumbersome, would increase individual cost per tip, exchanging entire hospital and surgery center inventories of suction tips, and would require re-engineering all tip designs in use today.

Currently, surgeons often kink the surgical tubing or clamp it to try to minimize suction noise. These methods are not practical and require the use of a second hand for tube manipulation prior to suctioning. This detracts attention from the surgery and adds time to the procedure. Clamping the tubing can cause bending/kinking would could increase likelihood of the tubing becoming obstructed with viscous fluids or debris.

### SUMMARY OF THE INVENTION

The present invention is directed to universal suction control devices that can be arranged in relation to surgical tubing to control the amount of air passing through the tubing. The suction control devices according to the present invention would be sterile and placed on or at the user end of the tubing, allowing for easy, point-of-care control of suction while minimizing excessive noise pollution from suction during surgery.

Some embodiments of the present invention can comprise an on/off control mechanism arranged to allow air to pass or to block air from passing through the surgical tubing. Some of these embodiments can be arranged in-line of surgical tubing used for suction during surgical procedures. Other embodiments can comprise finer control of the air passing through the tubing to vary the air passing through between fully blocked and fully open. The devices according to the present invention can have holding or locking mechanisms to hold the desired position with the desired amount of air passing through.

One embodiment of a suction control device according to the present invention comprising a mounting mechanism for mounting on surgical suction tubing. A first blocking portion is included for engaging the suctional tubing to block the passage of suction air through the tubing. A control surface is also included that is movable to control the engagement of said blocking portion with said surgical suction tubing to control the amount of air passing through said surgical suction tubing.

These and other aspects and advantages of the invention will become apparent from the following detailed description and the accompanying drawings, which illustrate by way of example the features of the invention.

### BRIEF DESCRIPTION OF THE DRAWINGS

FIG. 1 shows one embodiment of a suction control device according to the present invention;

FIG. 2a shows another embodiment of a suction control device according to the present invention in the off position;

FIG. 2b. shows the suction control device of FIG. 2a in the on position;

FIG. 3a is a perspective view of another embodiment of a suction control device according to the present invention;

FIG. 3b is one end view of the suction control device shown in FIG. 3a;

FIG. 3c is another end view of the suction control device shown in FIG. 3a.

FIG. 3d is a perspective view of the suction control device shown in FIG. 3a mounted on surgical tubing;

FIG. 3e is a side view of the suction control device shown in FIG. 3a mounted on surgical tubing;

FIG. 4 is a perspective view of one embodiment of a retainer for holding the tubing in the suction control device shown in FIG. 3a;

FIG. 5a is a side view of another embodiment of a suction control device according to the present invention;

FIG. 5b is a perspective view of the suction control device shown in FIG. 5a mounted on surgical tubing;

FIG. 5c is a side view of the suction control device shown in FIG. 5a mounted on surgical tubing;

FIG. 5d is a side view of the suction control device shown in FIG. 5a mounted on surgical tubing;

FIG. 5e is an end view of the suction control device shown in FIG. 5a mounted on surgical tubing;

FIG. 5f is opposite end view of the suction control device shown in FIG. 5a mounted on surgical tubing;

FIG. 6a is an end view of one embodiment of a mounting mechanism for holding surgical tubing in the suction control device shown in FIGS. 5a-5f; and

FIG. 6b is another end view of the mounting mechanism shown in FIG. 6a.

### DETAILED DESCRIPTION OF THE INVENTION

The present invention is described herein with reference to certain embodiments, but it is understood that the invention can be embodied in many different forms and should not be construed as limited to the embodiments set forth herein.

The present invention is directed to a simple, sterile, in-line, suction control device mounted to engage surgical suction tubing to control the amount of air passing through the tubing. The lower the amount of air being drawn through the suction tubing, the less suction noise that is generated at the tubing. The devices according to the present invention not only provide the user with finer control of the force of suction, it can also be used to reduce noise during surgery. Different embodiments can comprise an on/off valve or button that can be controlled by the operator during surgery, while other embodiments comprise a control that allow the operator to adjust to different levels of suction from full suction to no suction. Different embodiments of the suction device can include a lock or hold feature to hold the device in the desired position and desired level of suction.

The different embodiments can be universally compatible with a variety of conventional suction tubes and suction tips. Some devices can be mounted in-line with said surgical tubing and can comprise male end that accommodates current standard surgical tubing. The end of the surgical tubing terminates in a female end that is consistent with current standard size and be able to accept all currently used suction tips. When in the open position, the valve would allow clear flow of air through the tubing, to minimize any obstruction from debris or congealed fluid that is suctioned.

The default position of the valve would be in the off position, ensuring that suction and therefore suction noise is only occurring when intended. Depressing the button on the valve would open the tubing and allow suction through. The excursion of the button as it is depressed is proportional to the amount of tubing opened by the valve, allowing for precise control of the flow of suction through the tube. This can help modulate suction used for small volumes or in sensitive areas. The button could also be placed in a locked position for constant/prolonged suctioning on demand and to avoid operator fatigue. All of this manipulation of the suction flow can be done with the same hand used to operate the suction tip, allowing for single-hand use and decreased distraction during surgery.

Other embodiments of the suction control devices according to the present invention can be mounted on the surgical tubing instead of in-line with the tubing. Some of these arrangements allow for control of the air through the tubing while not interfering with or being between direct connection of the suction tips to the suction tubing. These embodiments are also easy to use with one hand and can allow for control of different levels of air through the suction tubing between fully open and fully blocked. These devices can also have a holding mechanism to hold the device at the desired amount of air passing through.

The present invention presents many advantages including but not limited to the following. The different embodiments can comprise a universal fit, negating the need to re-design or manufacture all current suction tips in use. The devices can also be easy to use, with some embodiments comprising a single hand control that can be operable with one hand and can be quickly and easily operated without looking at the device. This allows the user to quickly and easily operate the device while maintaining attention on the surgical area. The different embodiments also allow for quick exchange of suction tips during the procedure, to provide versatility is different suctioning uses. Different embodiments can have a default off position that automatically returns the device to off so that air does not pass through the tube. This minimizes suction noise pollution in the operating room. Other embodiments allow for control of flow through suction to control the amount of suction air through the tube and can be arranged to hold the control in the position to allow the desired air through. The different embodiments comprise a simple design that allows for inexpensive manufacture, low waste, ease of storage. The devices can be implemented in all operating rooms now without need for buying/changing other equipment

The present invention has wide industrial applicability. According to the United States Department of Health and Human Services, as of 2010 there were an estimated 53 million outpatient procedures and 46 million inpatient procedures performed in the United States annually. The majority of which will use suction of some sort. This figure doesn't include instances where suction is used outside of the operating room in a healthcare setting or surgeries performed internationally. This device therefore has broad applicability across all surgical specialties and settings.

It will be understood that when an element is referred to as being “on”, “connected to”, “coupled to”, or “in contact with” another element, it can be directly on, connected or couple to, or in contact with the other element or intervening elements may be present. In contrast, when an element is referred to as being “directly on”, “directly connected to”, “directly coupled to”, or “directly in contact with” another element, there are no intervening elements present.

Although the terms first, second, etc. may be used herein to describe various elements or components, these elements or components should not be limited by these terms. These terms are only used to distinguish one element or component from another element or component. Thus, a first element or component discussed below could be termed a second element or component, regions without departing from the teachings of the present invention.

Embodiments of the invention are described herein with reference to certain view illustrations that are schematic illustrations of embodiments of the invention. As such, the actual thickness of components can be different, and variations from the shapes of the illustrations as a result, for example, of manufacturing techniques and/or tolerances are expected. Embodiments of the invention should not be construed as limited to the particular shapes of the regions illustrated herein but are to include deviations in shapes that result, for example, from manufacturing. Thus, the regions illustrated in the figures are schematic in nature and their shapes are not intended to illustrate the precise shape of a region of a device and are not intended to limit the scope of the invention.

It is also understood that when an element such as a layer, region or substrate is referred to as being “on” another element, it can be directly on the other element or intervening elements may also be present. Furthermore, relative terms such as “inner”, “outer”, “upper”, “above”, “lower”, “beneath”, and “below”, and similar terms, may be used herein to describe a relationship of one layer or another region. It is understood that these terms are intended to encompass different orientations of the device in addition to the orientation depicted in the figures.

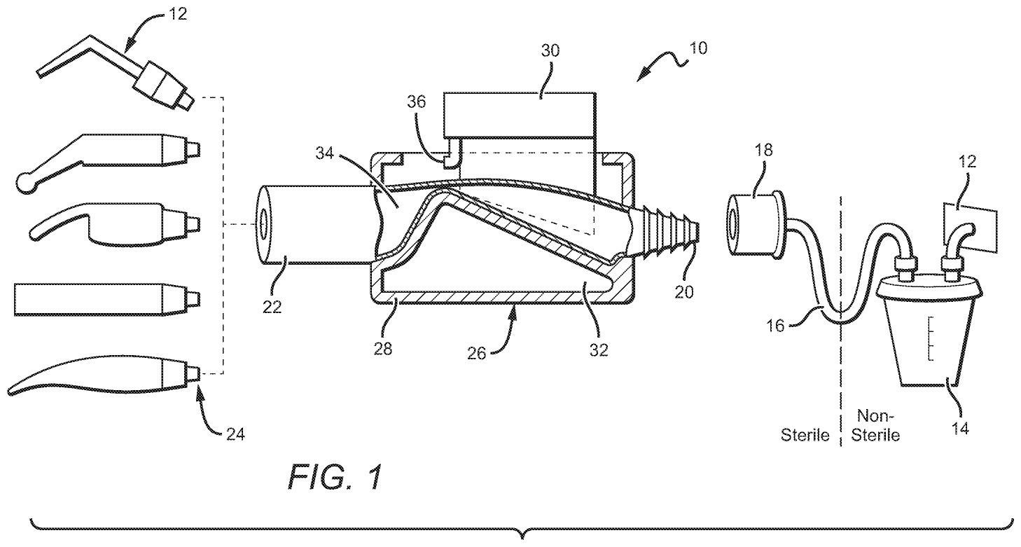

FIG. 1 shows one embodiment of a suction control device 10 according to the present invention. The device 10 is coupled to suction source 12 that can either be a standard wall suction or local machine suction and are generally known. A suction canister 14 is coupled to the suction source and is arranged to capture liquids or solids traveling that are captured during suctioning. These are also generally known. Surgical suction tubing 16 is also coupled to the cannister 14, with the tubing being standard industry sized tubing such as 3/16 inch tubing. The surgical area (or surgical field) can be divided between sterile and non-sterile sections. A section of the surgical tube 16 can be in the sterile portion while a different section can be in the non-sterile portion.

The tubing 16 comprises a female connector (female end) 18 opposite the cannister 14 and can comprise an industry standard connector to attach to different suction tips. The suction control device 10 is provided with a male connector 20 that mates with the female connector 18 for mounting the device 10 to the tubing 16. At the opposite end, the on demand suction device 10 has a female connector 22 is sized for the male connector on standard suction tips 24. Between the male and female connectors 22, 24 an On/Off button mechanism 26 is provided to turn on and off the flow of air through the suction device. This arrangement is referred to as in-line mounting of the device 10 between the tubing 16 and suction tips 24.

Many different On/Off mechanisms can be used in different embodiments of the present invention. In the embodiment shown, the device 10 comprise a mechanism housing 28 that holds the working components to control the air flow through the device 10. The housing 28 holds an On/Off button 30 that is operable to start and stop the flow of air through the tubing 18. A compressive coil 32 is arranged within the housing 28 and can comprise a tensioned coil similar in shape to a mousetrap hammer. Compressible tubing 34 is also arranged in the housing and has properties that allow it to be easily compressed by the coil 32, while having sufficient memory to open spontaneously when the coil 32 is released. The tubing 34 also has wall stiffness to not occlude under the negative pressure of the suction. The tubing 34 can be made of many different materials or combinations of materials, with some tubing being made silicone. The coil 32 is positioned so that it compresses the portion of tubing contained within the housing 28 occluding suction.

The suction device 10 as shown is arranged such that when the button 30 is not compressed, the coil 32 is free to compress the tubing 34 to block air through the device 10. To allow air to pass, the button 30 is compressed, which in turn compresses the coil 32 away from the tubing, allowing air to pass. The housing 28 and button 30 can also comprise a locking mechanism 36 to hold the button 30 down to allow air to pass. In the embodiment shown the lock mechanism can be activated by depressing and then sliding the button 30 forward. This allows the device 10 to continue to allow air to flow while not requiring continued downward pressure on the button 30. The button 30 can return to its default off position by sliding the button 30 back and removing downward pressure from the button 30.

It is understood that other embodiments can be arranged so that the default is the On position with air flowing. It is further understood that other embodiments can have multiple position control to vary the amount of air passing through the device 10 between fully on and fully blocked.

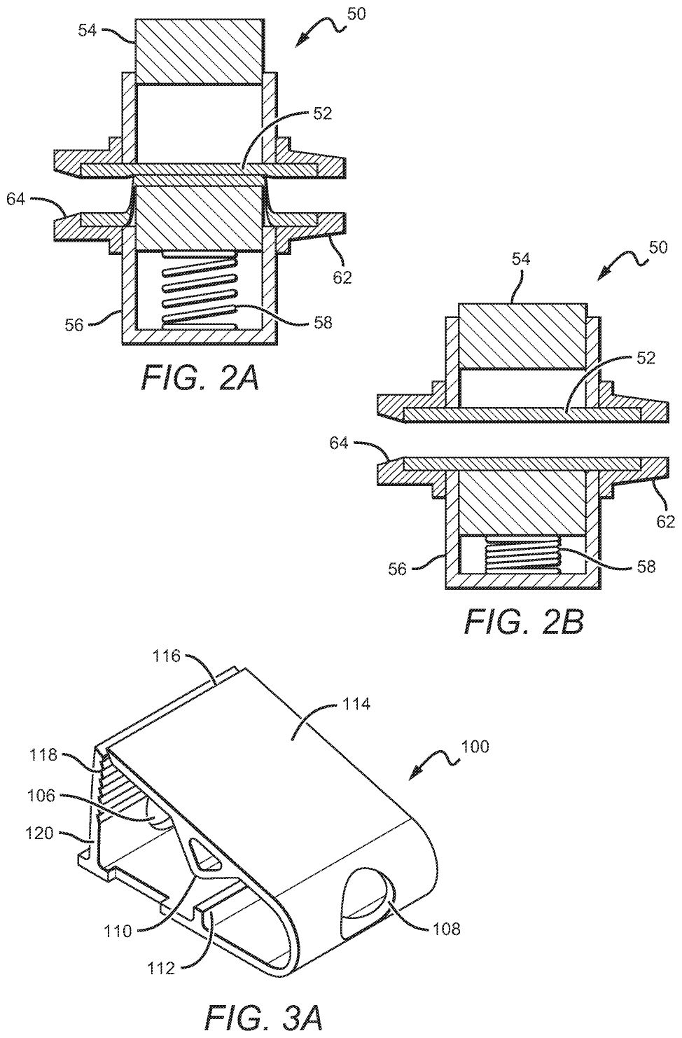

FIGS. 2a and 2b show another embodiment of a suction control device 50 according to the present invention, with this embodiment referred to as a variant compression device or “Cord-Lock Style” on/off mechanism. The control device 50 uses a mechanism similar to that on drawstring cord locks, with this variant compressing the suction tubing 52 between first and second concentric cylinders 54, 56 with offset eyelets. FIG. 2a shows the device 50 in the closed/off position, and FIG. 2b shows the device 59 in the open/on position.

In the embodiment shown, the first (inner) cylinder 54 can also function as the on/off button with the top of the cylinder functioning as the surface to depress to control the flow of air off. The base has a hollow canal for the suction tubing to pass through. The second outer cylinder 56 can also act as the device housing can contain the compressive spring 58, locking mechanism (not shown) and male and female connectors 62, 64.

Like the embodiment above, the device 50 has a default position of off with air blocked from passing through. When the button 55 is in the up and off position, the spring 58 is free to compress the tubing 66. When the button 55 is compressed, the spring 58 is also compressed, with opens the tubing for air to flow. The device 50 can also comprise a locking mechanism that can hold the button in the on position for prolonged use. Possible mechanisms include a slide lock or a cycling tooth lock similar to that used in retractable pens.

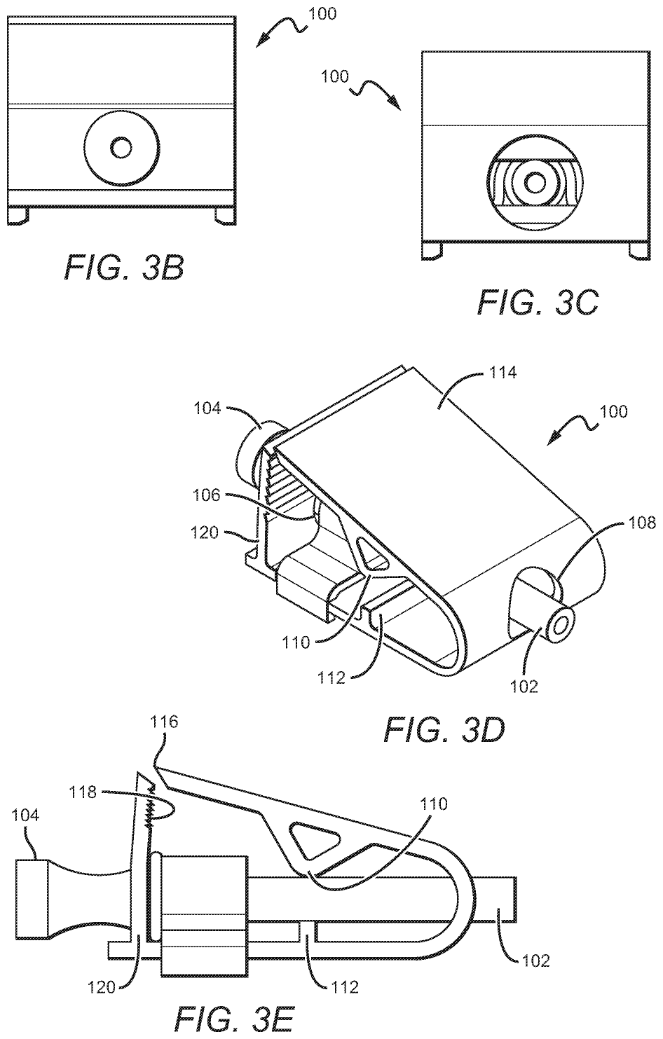

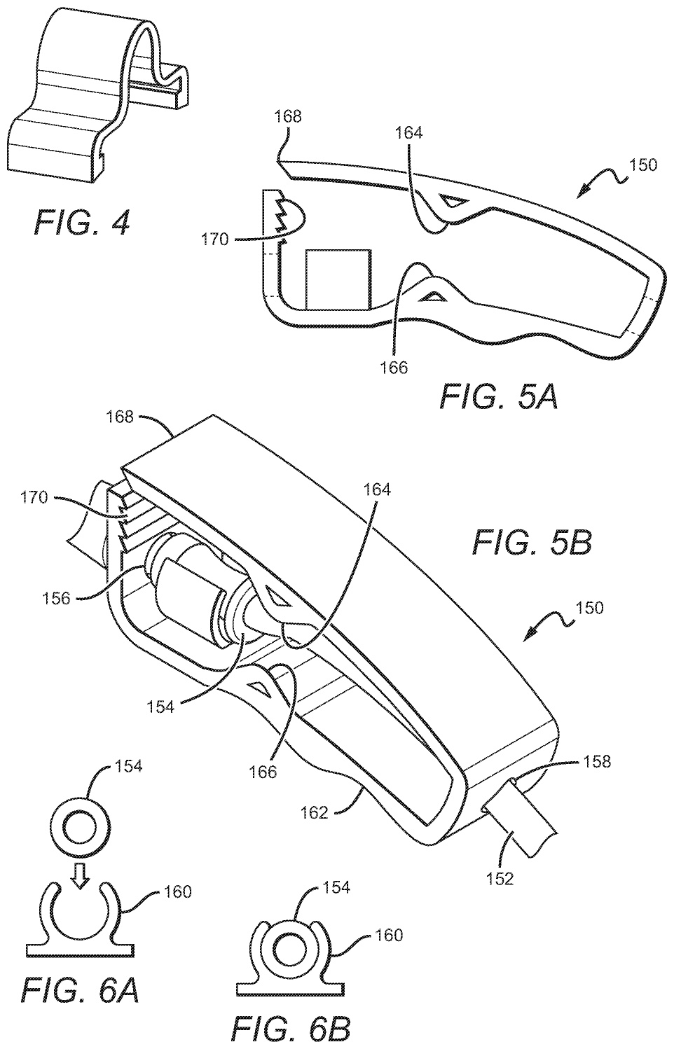

The devices described above generally comprise in-line devices that are placed between the surgical tubing and the suction tip. Other embodiments can comprise mechanism mounted on the surgical tubing. FIGS. 3a-3g show another embodiment of a suction control device 100 according to the present invention that is mounted directly on the surgical tubing 102, with the embodiment shown being mounted adjacent the female connector 104. The device 100 is arranged such that it does not interfere with, and is not between, the connection between the female connector 104 and connection with the male connector (not shown) of a suction tip.

The device 100 is generally wedge-shaped and has first and second holes 106, 108 that allow the tubing to pass through the device 100. The female connector 104 is held near the first hold 106, and the surgical tubing 102 passes through the second hold 108. The tubing 102 is held can be held in the device 100 using different mechanisms, with the embodiment shown using a retainer (described below) that mounts to the device 50 to firmly holds the tubing 102 to the inside surface of the device 100.

The top inside surface of the device 100 comprises a raised blocking portion 110, with the bottom inside surface of the device 100 comprising an opposing blocking potion comprising a ridge 112. The blocking portion 110 and blocking ridge 112 cooperate to block air from passing through the tube during operation of the device 100. As the blocking portion 110 is brought closer the blocking ridge 112 the tube 100 is pinched and compressed between the two. This causes the tubing to collapse to decrease or fully block air from passing thought the tube 102.

The devices described above were arranged to operate in either the on or off positions, with air flowing through the tube or air fully blocked from passing through. The device 100 is arranged for greater control to fully allow air to pass, to partially block air from passing through the tube to reduce the amount of air passing through, or to fully block air from passing through.

To operate the device 100, the control surface 114 is moved down to bring the blocking portion 110 closer to the ridge 112. The edge of the control surface 114 closest to the female connector 104 has a lip 116 that interacts with a sawtooth portion 118 on the devices lower section 120. Lip 114 engages the teeth of the sawtooth portion 118 as the control surface is moved toward down. The control surface 114 is held in the particular downward position by this engagement, and each engagement moving down represents a different control position for controlling a differing amount of air passing through the tubing. The engagement of the lip 116 with the topmost of the teeth in the sawtooth portion 118 would position the blocking portion 110 and ridge 112 so there is little blockage of the tubing. The engagement of the lip 116 with the lowest of the teeth in the sawtooth portion would position the blocking portion 118 and ridge 110 closer such that air would be blocked from passing through the tube. The resolution of control of air through the tube 102 depends on the number of teeth in the sawtooth portion 118.

The engagement of the lip 114 with the sawtooth portion can be released by the user pushing the sawtooth portion 118 in a direction away from the lip 116. This causes the lower portion 120 to flex away from the control surface 114, causing the lip 116 to disengage from the sawtooth portion 118. This allows the control surface to move back to its default upmost position, moving the blocking portion 110 away from the ridge 112. This in turn allows air to fully pass through the tube 102.

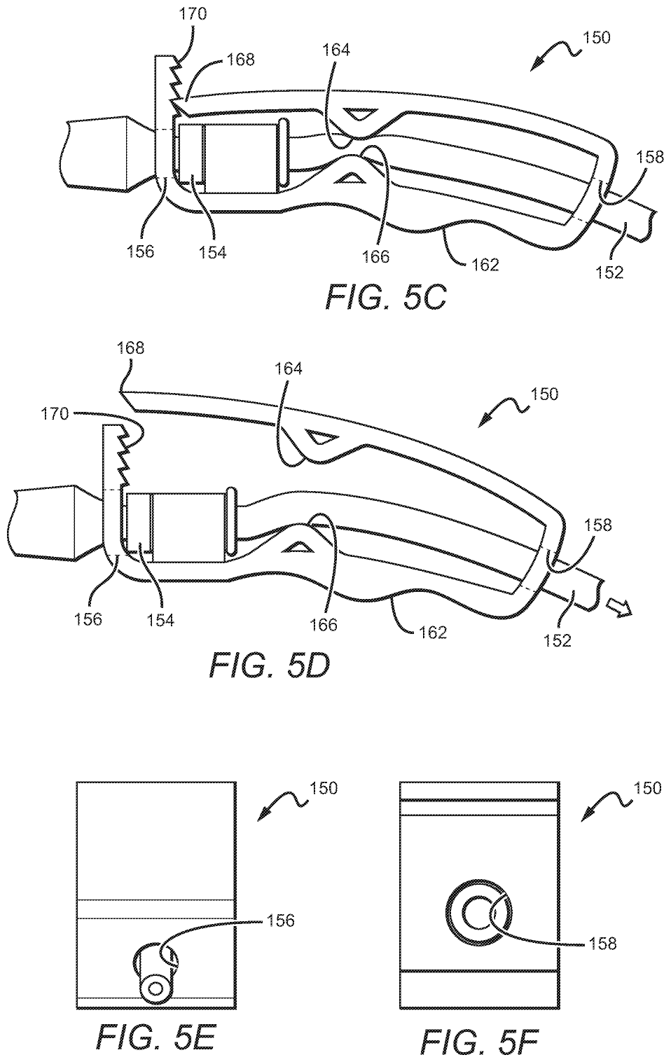

FIGS. 5a-5f show another embodiment of suction control device 150 according to the present invention that operates in a manner similar to the device 100 described above. The device 150 is arranged to be mounted on the tubing 152 near the tubing's female connector 154. The tubing 152 passes through the device 150, through first and second holes 156, 158. In this embodiment, the tubing 152 is held to the device 150 at a U clip 160 (best shown in FIGS. 6a and 6b) sized so that the female connector 154 is snapped in place and held firmly within the clip 160. In this embodiment, the clip 160 is molded as part of the device 150, resulting in ease of manufacturing and use. It is understood that other mounting mechanisms can be used such as tongue and slot arrangements, buckles, hinges or external straps. The device 150 does not interfere with the connection of the female connector 154 with the corresponding male connectors from suction tips.

The bottom surface 162 of the device 150 has an ergonomic shape to be comfortably held in the user's hand and is arranged so that the control surface 164 can be operable by the user's thumb. The tubing 152 runs between first and second blocking portion 164, 166 within the device 150. The control surface 164 can be moved down during operation, which moves the blocking portion 164, 166 closer together. This causes the blocking portions to collapse the tubing 152 with the amount of blocking directly corresponding to the distance that the control surface is moved down.

Like the device 100, the control surface has a lip 168 that engages with a sawtooth portion 170 as the control surface is moved down. The control surface can be held in the desired position by this engagement, and the user can release the device 50. As best shown in FIG. 5e, when the lip is held in the lowest position on the sawtooth portion 170, the blocking portion collapses the tubing fully blocking air from passing through the tubing 152. To release the engagement of the lip 168 and sawtooth portion 170, pressure is applied to the device 150 at the sawtooth portion 170, causing it to flex away from the control surface 164. This causes the lip 168 to disengage from the sawtooth portion 170. With this disengagement and downward pressure removed, the device 150 is arranged so that the control surface returns to the up position as shown in FIG. 5d. This is the position of the device 150 that allows air to pass through the tubing 152.

The different features of the suction control devices according to the present invention can be made of many different materials manufactured using many different methods. Some embodiments can be made of plastics and can be manufactured using standard injection molding manufacturing processes.

Is also understood that the suction control device described above are controlled by the user's hand, but other embodiments can be controlled in different ways such as by foot control. In still other embodiments, the suction control devices can be arranged to be voice controlled or wirelessly controlled, such as by Blue Tooth communication.

Although the present invention has been described in detail with reference to certain preferred configurations thereof, other versions are possible. Therefore, the spirit and scope of the invention should not be limited to the versions described above.

We claim:

1. A suction control device, comprising:
a mounting mechanism for mounting on surgical suction tubing;
a first blocking portion for engaging said suctional tubing to block the passage of suction air through said tubing; and
a control surface movable to control the engagement of said blocking portions with said surgical suction tubing to control the amount of air passing through said surgical suction tubing.

2. The suction control device of claim 1, wherein said control surface comprises an On/Off button.

3. The suction control device of claim 1, having a holding mechanism to hold said control surface at the desired location with the desired engagement of said blocking portion with surgical suction tubing.

4. The suction control device of claim 3, wherein said holding mechanism comprise a lip engaging a sawtooth surface.

5. The suction control device of claim 4, wherein said sawtooth surface is disengaged from the said lip to release said holding of said control surface.

6. The suction control device of claim 1, further comprising a second blocking portion, wherein said surgical tubing is between said first and second blocking portions.

7. The suction control device of claim 1, wherein said first blocking portion is formed integral to said control surface.

8. The suction control device of claim 1, wherein said mounting mechanism comprises a C-clamp.

9. The suction control device of claim 1, wherein said first blocking portion comprises a compressive coil.

10. The suction control device of claim 1, wherein said first blocking portion comprises a spring.

11. A suction control device, comprising:
a mounting mechanism for mounting on surgical suction tubing;
at least one blocking portion;
a control surface movable to move said at least one blocking portion in relation to said surgical tubing to engage and collapse said surgical tubing to vary the amount of air passing through said surgical suction tubing; and
a holding mechanism to hold said control surface at the desired position with the desired level of engagement with said surgical tubing.

12. The suction control device of claim 11, wherein said at least one blocking portion partially collapses said surgical tubing to reduce the amount of air passing though said surgical tubing.

13. The suction control device of claim 11, wherein said at least one blocking portion fully collapse said surgical tubing to block air from passing through said surgical tubing.

14. The suction control device of claim 11, wherein said holding mechanism comprises a lip on said control surface engaging a sawtooth surface.

15. The suction control device of claim 14, wherein said sawtooth surface is disengaged from the said lip to release said holding of said control surface.

16. The suction control device of claim 1, wherein said at least one blocking surface comprises a first and said blocking portions with said surgical tubing running between.

17. A suction control device, comprising:
a mounting mechanism for mounting on surgical suction tubing;
at least one blocking portion;
a blocking element arranged adjacent to said surgical tubing to engage and collapse said surgical tubing to block air from passing through said tubing;
an On/Off button positioned to either to disengage said blocking element from tubing to allow air to fully pass, or to allow said blocking element to fully engage said tubing to block substantially all air from passing though said tubing.

18. The suction control device of claim 17, wherein said blocking element comprises a compressive spring.

19. The suction control device of claim 17, arranged for mounting in-line an end of said surgical tubing and a suction tip.

20. The suction control device of claim 17, wherein said On/Off button comprises a cord-lock mechanism.
